(19) United States
(12) Patent Application Publication
(10) Pub. No.: US 2004/0224006 A1
(43) Pub. Date: Nov. 11, 2004

(54) Ancrod irradiated, impregnated or coated sutures and other first aid or wound management bandaging materials for minimizing scarring and/or preventing excessive scar formation

(76) Inventor: Samn Raffaniello, East Durham, NY (US)

Correspondence Address: HESLIN ROTHENBERG FARLEY & MESITI PC, 5 COLUMBIA CIRCLE, ALBANY, NY, 12203, US

(21) Appl. No.: 10/829,143

(22) Filed: Apr. 21, 2004

Related U.S. Application Data
(60) Provisional application No. 60/464,229, filed on Apr. 21, 2003.

(51) Int. Cl. A61K 38/48; A61K 31/515; A61K 31/19

(52) U.S. Cl. 424/445; 424/94.64; 514/557; 514/571; 514/270

(57) ABSTRACT

A method for minimizing scarring and preventing excessive scar formation at an injury site is disclosed. The method involves the topical and/or local application of a therapeutically effective amount of a defibrinogenating agent or of a fibrinolytic agent that may be delivered in an appropriate vehicle in a controlled- or timed-release manner. In accordance with the principles of the invention, the defibrinogenating agent or fibrinolytic agent is applied as a coating on, or is irradiated or impregnated into or onto a delivery vehicle such as, for example, sutures, dissolvable sutures, bandages, gauze pads, or other types of first aid bandaging materials. Such application may take the form of a controlled- or timed-release aspect of either the vehicle, the delivery material or the therapeutic agent, such that the release of the therapeutic agent may be regulated to produce an appropriate therapeutic pattern or defibrinogenation or fibrinolysis. In a preferred aspect of the invention, the defibrinogenating agent is ancrod, and the mode of application is as ancrod-coated sutures.

### FIELD OF THE INVENTION

The present invention relates to the use of a defibrinogenating agent, such as ancrod and fibrinolytic agents irradiated, impregnated or coated in or on suture material and other first-aid bandaging materials for the minimization of scarring and the prevention of excessive topical and/or local scar formation.

### BACKGROUND OF THE INVENTION

Fibrinogen is a protein that is a precursor to fibrin formation. Fibrin is a protein that initiates blood clots at wound sites. Fibrinogen and fibrin are believed to play key roles in scar formation at the injury site. Inflammation is the normal acute reaction of the tissues after any injury. The immediate response of the blood supply to the area is a nervous constriction of the vessels. This is followed immediately by vasodilation that allows fluid to exit the capillaries and flood the area. The fluid, plasma, contains fibrinogen which is cleaved to form fibrin strands that form substantial portions of the blood clot. Eventually, the clot is replaced by granulation tissue, a connective tissue with a rich blood supply. Collagen and ground substance (proteoglycans) are produced by the fibroblasts within the granulation tissue, and a scar forms. Defibrinogenating agents, which function to reduce or remove circulating fibrinogen, which converts to fibrin at the injury site, as well as fibrinolytic agents, which act directly to deplete fibrin, represent a new strategy for minimizing scarring and/or preventing excessive scarring.

By removing the clotting precursor, fibrinogen, from the injury site, a reduction or alteration in fibrin formation and hence fibrin deposition is seen. Controlled, patterned defibrinogenation can be used as a strategy for controlling the timing, pattern and amount of fibrin deposition occurring at an injury site in a way that allows for sufficient normal fibrin deposition in the early stages of healing, thereby promoting the initiation of the scarring process, and then controlling further fibrin deposition to control, reduce or minimize the extent of scarring as the process continues to evolve. Since ancrod, as the preferred method, does not cause the lysis of normal clots already formed, the initial fibrin deposition to the wound site prior to the introduction of ancrod as a defibrinogenating agent, is not adversely affected and ancrod administration will not reverse the positive effects of early fibrin deposition.

A particularly effective defibrinogenating agent is Empire Pharmaceutical's brand of ancrod (VIPRINEX®, under license from Abbott Laboratories Chicago, Ill. USA), a biological derived from the venom of the Malayan pit viper. The agent consists of a glycosylated 234-amino acid protein.

Ancrod specifically functions by interfering with the fibrinogen to fibrin conversion. It has a thrombin-like action with substrate specificity for fibrinogen, while lacking any effect on Factor XIII, other coagulation factors or platelets. Any fibrin polymers that do arise, are rapidly digested by plasmin and eliminated from circulation via the reticulo-endothelial system. The pharmacological consequence of this action is the depletion of plasma fibrinogen and the reduction of erythrocyte aggregation on blood viscosity. The endogenous fibrinolytic system is strongly activated; indicated by a rise in fibrin degradation products and other clear indicators of plasmin-mediated fibrinolysis.

The feasibility of various routes of administration of fibrinolysis-enhancing agents for the prevention of surgical adhesions is described in U.S. Pat. No. 6,461,640.

### SUMMARY OF THE INVENTION

In accordance with the principles of the present invention, Applicant has discovered that the direct application or controlled- or timed-release local or topical administration of a therapeutically effective amount of a defibrinogenating agent, such as, for example, ancrod, urokinase, streptokinase and anticonvulsants such as, for example, phenobarbital or valproic acid, provides effective treatment for minimizing or preventing excessive topical and or local scarring at an injury site.

In one aspect, therefore, the invention relates to a method for minimizing scarring and/or preventing excessive scar formation at an injury site, the method comprising applying to the injury site a first aid bandaging material that has been coated, irradiated or impregnated with a therapeutically effective amount of a defibrinogenating agent. The first aid bandaging material may be any type of bandage or gauze pad. The defibrinogenating agent is chosen from the group consisting of ancrod, urokinase, streptokinase, phenobarbital and valproic acid.

In another aspect, the invention relates to a method for minimizing scarring and/or preventing excessive scar formation at an injury site, the method comprising applying to the injury site a first aid bandaging material that has been coated, irradiated or impregnated with, a therapeutically effective amount of a fibrinolytic agent. In this embodiment of the present invention, the fibrinolytic agent is chosen from the group consisting of tissue-plasminogen activator (t-PA), recombinant tissue-plasminogen activator (rt-PA), advance-generation fibrates, such as fenofibrate and fibrinolytic derivatives of recombinant tissue-plasminogen activator, such as reteplase (rPA), lanoteplase (nPA) and tenecteplase (TNK-tPA).

The invention further provides for the minimization of external scarring caused by wounds and/or incisions and/or by the use of sutures in routine wound closure procedures or in the closing of surgical incisions postoperatively, or any such locally or surgically invasive procedure where scarring may develop at an incisions, wound or sutured site.

In a related aspect, therefore, the present invention relates to a method for minimizing scarring and/or preventing excessive scar formation at an injury site, the method comprising the use of sutures or dissolvable sutures to close the wound site, wherein said sutures or dissolvable sutures have been coated, irradiated or impregnated with a therapeutically effective amount of a defibrinogenating agent such as ancrod, urokinase, streptokinase, phenobarbital and valproic acid or defibrinlytic agent such as tissue-plasminogen activator (t-PA), recombinant tissue-plasminogen activator (rt-PA), advance-generation fibrates, such as fenofibrate and fibrinolytic derivatives of recombinant tissue-plasminogen activator, such as reteplase (rPA), lanoteplase (nPA) and tenecteplase (TNK-tPA.

### DETAILED DESCRIPTION OF THE INVENTION

All patents, applications, publications and other references cited herein are hereby incorporated by reference in their entirety into the present application.

The invention applies to the treatment of any prospective site of scarring, irrespective of the potential degree of scarring severity, and may include, but is not limited to, applications involving the potential for excessive scarring in the form of hypertropic scars, for example.

In a preferred aspect of the invention, the defibrinogenating agent is ancrod, available commercially, for example, under the trade names ARVIN® or VIPRINEX® (Empire Pharmaceuticals, Inc., New York, USA).

As used herein, the term “ancrod” encompasses not only products prepared from the ancrod protease isolated from snake venom, but also any products containing ancrod proteins obtained through genetic manipulation.

Methods for the preparation of ancrod from snake venom are well known, and include, but are in no way limited to, the methods taught in U.S. Pat. Nos. 6,200,791; 3,743,722 and 3,879,369; Great Britain Patent documents 1,094,301; 1,177,506 and 1,293,793; and German patent documents 2,428,955 and 2,734,427. Methods for the preparation of ancrod products through genetic manipulation are taught, for example, in U.S. Pat. No. 6,015,685.

As a further aspect of the invention, Applicant has discovered that fibrinolytic agents such as tissue-plasminogen activator (tPA), recombinant tissue-plasminogen activator (rt-PA), fibrinolytic derivatives of recombinant tissue-plasminogen activator, such as, for example, reteplase (rPA), lanoteplase (nPA) and tenecteplase (TNK-tPA), as well as various advance-generation fibrates such as hypocholesterolemic drugs, such as, for example, fenofibrate, also effectively eliminate fibrinogen from blood, thereby interfering with the formation of fibrin and any resulting scarring.

According to the principles of the invention, the defibrinogenating or fibrinolytic agent may be administered to the wound site in the form of a pharmaceutical dosage unit for local administration. Such dosage unit may take the form of a controlled- or timed-release aspect of either the vehicle, the delivery material or the therapeutic agent, such that the release of the administered agent may be regulated to produce an appropriate therapeutic pattern of defibrinogenation or fibrinolysis. The pharmaceutical may, for example, be in the form of sutures, dissolvable sutures, bandages, gauze pads or any other topical first aid bandaging materials, any of which have been coated, irradiated or impregnated with the defibrinogenating or fibrinolytic agent of choice. Preferably, the pharmaceutical dosage unit will be in the form of ancrod-coated, irradiated or impregnated sutures (SCARLESS SUTURES™, Empire Pharmaceuticals, Inc., New York, USA).

To determine if ancrod applied locally to a sutured wound exerts a systemic defibrinogenating effect and to evaluate the influence of locally applied ancrod on wound healing and scarring, the following study is performed.

Study Design

Four groups of six rats are studied (24 rats total). Prior to the study, baseline fibrinogen levels are measured in each rat. Each rat receives two identical surgically-induced abdominal wounds which are then sutured. Prior to treatment with a defibrinogenating agent, fibrinogen levels are then measured at one and two-hours post-suturing. Subsequently, the animals are treated as follow:

Group A: IV ancrod is administered to each animal

Group B: Ancrod is applied locally to one wound and the other wound is untreated

Group C: Ancrod is applied locally to both wounds

Group D: No treatment is administered

Fibrinogen levels are then measured at 1, 2, 3, 4, 6 and 9 hours post-treatment.

Evaluation

Fibrinogen levels are charted and compared between groups. Wounds are evaluated for dehiscence and bleeding throughout the study, and the time and duration of each event in relation to the treatment is recorded. Wounds/scars are photographed and evaluated for the following parameters at 3, 7, 10 and 14 days post-suturing, with comparisons being made between scars in each animal and between scars in groups of animals:

Overall appearance

Size

Color

Texture

Intensity

Fading

1. A method for minimizing scarring and/or preventing excessive scar formation at an injury site, the method comprising applying to the injury site a first aid bandaging material that has been coated, irradiated or impregnated with a therapeutically effective amount of a defibrinogenating agent.

2. The method of claim 1 wherein said first aid bandaging material is a bandage or gauze pad.

3. The method of claim 1 wherein said defibrinogenating agent is chosen from the group consisting of ancrod, urokinase, streptokinase, phenobarbital and valproic acid.

4. A method for minimizing scarring and/or preventing excessive scar formation at an injury site, the method comprising applying to the injury site a first aid bandaging material that has been coated, irradiated or impregnated with, a therapeutically effective amount of a fibrinolytic agent.

5. The method of claim 4 wherein said fibrinolytic agent is chosen from the group consisting of tissue-plasminogen activator (t-PA), recombinant tissue-plasminogen activator (rt-PA), advance-generation fibrates and a fibrinolytic derivative of recombinant tissue-plasminogen activator.

6. The method of claim 5 wherein said fibrinolytic derivative of recombinant tissue-plasminogen activator is chosen from the group consisting of reteplase (rPA), lanoteplase (nPA) and tenecteplase (TNK-tPA).

7. The method of claim 5, wherein said advance-generation fibrate is fenofibrate.

8. A method for minimizing scarring and/or preventing excessive scar formation at an injury site, the method comprising the use of sutures or dissolvable sutures to close the wound site, wherein said sutures or dissolvable sutures have been coated, irradiated or impregnated with a therapeutically effective amount of a defibrinogenating agent.

9. The method of claim 8 wherein said defibrinogenating agent is chosen from the group consisting of ancrod, urokinase, streptokinase, phenobarbital and valproic acid.

10. The method of claim 9 wherein said defibrinogenating agent is ancrod.

11. A method for minimizing scarring and/or preventing excessive scar formation at an injury site, the method comprising the use of sutures or dissolvable sutures that have been coated, irradiated or impregnated with a therapeutically effective amount of a fibrinolytic agent, to close the wound site.

12. The method of claim 11 wherein said fibrinolytic agent is chosen from the group consisting of tissue-plasminogen activator (t-PA), recombinant tissue-plasminogen activator (rt-PA), fibrinolytic derivatives of recombinant tissue-plasminogen activator, advance-generation fibrates.

13. The method of claim 12 wherein said fibrinolytic derivative of recombinant tissue-plasminogen activator is chosen from the group consisting of reteplase (rPA), lanoteplase (nPA) and tenecteplase (TNK-tPA).

14. The method of claim 11 wherein said fibrate is fenofibrate.
